(19) United States
(12) Patent Application Publication
(10) Pub. No.: US 2012/0082648 A1
(43) Pub. Date: Apr. 5, 2012

(54) Colonization factor (CF) antigens of enterotoxigenic escherichia coli in recombinant bacteria

(76) Inventors: Michael Lebens, Hokerum (SE); Ann-Mari Svennerholm, Vastra Frolunda (SE); Joshua Tobias, Goteborg (SE)

(21) Appl. No.: 13/136,996

(22) Filed: Aug. 15, 2011

Related U.S. Application Data
(63) Continuation of application No. 12/218,967, filed on Jul. 18, 2008.
Application No. 12/218,967 is a continuation of application No. PCT/SE2007/050051, filed on Jan. 31, 2007.
(60) Provisional application No. 60/763,905, filed on Feb. 1, 2006.

(51) Int. Cl. A61K 35/74; C12N 15/70; A61P 1/12; C12N 1/21

(52) U.S. Cl. 424/93.2; 435/252.33; 435/488

(57) ABSTRACT

Escherichia coli strains, such as enterotoxigenic E. coli strains, genetically engineered to express from recombinant plasmids one or more colonization factors (CFs) associated with enterotoxigenic Escherichia coli bacteria (ETEC) in an increased amount compared to said CFs expressed by ETEC wild-type reference strains, as well as a method of producing such strains, and vaccine compositions against diarrhea comprising such strains, are described. Further, E. coli strains expressing unnatural combination of at least two different CFs, e.g., CFA/I+CS2, CFA/I+CS6, or CS2+CS4 are disclosed.

### CROSS-REFERENCE TO RELATED APPLICATIONS

This application is a continuation of co-pending U.S. patent application Ser. No. 12/218,967, filed Jul. 18, 2008, which application is a continuation of PCT International Patent Application No. PCT/SE2007/050051, filed Jan. 31, 2007, designating the United States of America, and published, in English, as PCT International Publication No. WO 2007/089205 A1 on Aug. 9, 2007, which application claims priority to U.S. Provisional Patent Application Ser. No. 60/763,905, filed Feb. 1, 2006, the entire contents of each of which are hereby incorporated herein by this reference. This application claims the benefit under 35 U.S.C. §119(e) to U.S. Ser. No. 60/763,905 filed on Feb. 1, 2006.

### TECHNICAL FIELD

The invention relates to recombinant bacterial cells, in particular, Escherichia coli cells, useful for vaccines or compositions against diarrhea. The E. coli cells express from recombinant plasmids one or more colonization factors (CFs) in increased amount(s). The recombinant plasmids enable expression from one bacterial cell of at least two different CFs that are not expressed in the same wild-type bacterial cell.

### BACKGROUND OF THE INVENTION

Enterotoxigenic Escherichia coli (ETEC) is a major cause of travelers' diarrhea and of diarrheal morbidity and mortality of children in endemic areas in many parts of the world. Virulence of the bacteria is associated with expression of fimbrial colonization factors (CFs) that mediate bacterial adhesion to the intestine and with secretion of heat-labile (LT) and/or heat-stable (ST) toxins, which by affecting electrolyte and fluid transport processes in the gut are responsible for the diarrhea characteristic of the disease (Gaastra and Svennerholm, 1996; Qadri et al., 2005a; Sanchez and Holmgren, 2005).

Protection against ETEC disease is associated with antibody-mediated neutralization of LT and immune responses against the CFs (Levine et al., 1994; Svennerholm and Holmgren, 1995; Svennerholm and Savarino, 2004). In general, the purpose of a vaccine is to induce an immune response in recipients that provides protection against subsequent challenge with the actual pathogen. This may be achieved by inoculation with a live attenuated strain of a pathogen, i.e., a strain having reduced virulence such that it does not cause the disease while still stimulating an effective immune response, or by administration of one or more killed strains of the pathogen that can elicit protective immune responses that are effective against infecting virulent strains. For immunization against enteric infections, the vaccine should preferably be given by the oral route to efficiently stimulate an effective immune response locally in the intestinal mucosa, but also other mucosal routes or parenteral or even transcutaneous routes may be used for inducing protective immunity.

Development of an effective vaccine that protects against disease caused by ETEC is difficult. More than 100 different serotypes have been associated with pathogenic strains. Furthermore, these strains can carry one or more of a large number of CFs (each of which is antigenically different) that facilitate the establishment of the infection in the intestine (Gaastra and Svennerholm, 1996).

Considerable evidence exists that immune responses directed against the CFs are protective and that mucosal immune responses in the intestine are of particular importance for protection (Svennerholm et al., 1988, 1990; Levine et al., 1994; Qadri et al., 2005). To induce such responses, an ETEC vaccine should preferably be administered orally.

An oral, killed, whole cell ETEC vaccine containing five strains representing common ETEC serotypes and expressing several of the most commonly encountered CFs (in several cases, usually referred to as coli surface (CS) proteins), i.e., CFA/I, CS1, CS2, CS3, CS4, and CS5, together with recombinant cholera toxin B subunit (CTB, which is highly homologous to the B subunit of ETEC LT) was previously developed. (Svennerholm and Holmgren, 1995; Svennerholm and Savarino, 2004). Initial clinical trials with this vaccine gave rise to significant immune responses against both CTB and the specific CFs present in the vaccine in Swedish volunteers and subsequently in adults and children in Egypt and Bangladesh (Jertborn et al., 1993; Åhren et al., 1998; Savarino et al., 1998, 1999; Qadri et al., 2005a, 2005b). The vaccine also provided significant protection against diarrhea sufficiently severe to interfere with the daily activity of American travelers going to Mexico and Guatemala (Sack et al., 2002; Svennerholm and Savarino, 2004). However, the protection efficacy of the vaccine in Egyptian infants 6 to 18 months of age was found to be low (Savarino et al., to be published). This suggested that whereas the vaccine was effective against more severe disease in travelers, it was not sufficiently potent to protect infants living in endemic areas (Svennerholm and Steele, 2004).

One of the reasons for the low efficacy of the described ETEC vaccine in infants is thought to be due to the comparatively low antibody responses found to the CF antigens in this age group (Savarino and Svennerholm, 2004). This poor response may be improved by giving higher doses of the different CFs and, hence, increasing the amount of these antigens in a vaccine dose, which is a priority for continued development of a killed ETEC whole-cell vaccine. It is not feasible to simply increase the number of ETEC bacteria administered with each vaccine dose since it has been shown that giving high amounts of inactivated E. coli bacteria (even of an E. coli K12 placebo preparation) to young children 6 to 18 months of age can result in adverse reactions in the form of vomiting, probably due to the large amounts of endotoxin (LPS). These adverse effects were not observed if a lower (three-fold lower) dose of bacteria was given (Qadri et al., 2005b; Savarino et al., to be published).

### DISCLOSURE OF THE INVENTION

Described is a bacterial strain, namely an Escherichia coli strain, genetically engineered to express from recombinant plasmids one or more colonization factors (CFs) associated with enterotoxigenic Escherichia coli bacteria (ETEC) in an increased amount compared to the CFs expressed by ETEC wild-type reference strains.

The E. coli strain is, e.g., a non-toxigenic E. coli strain and the CFs are, e.g., selected from the group consisting of CFA/I, CS1, CS2, CS3, CS4, CS5 and CS6.

The invention comprises an E. coli strain that expresses an unnatural combination of at least two different CFs, such as a strain that expresses an unnatural combination of two different CFs selected from, but not limited to, the group consisting of CFA/I+CS2, CFA/I+CS6 and CS2+CS4.

An E. coli strain according to the invention may be a strain that does not express an antibiotic resistance gene. Further, the E. coli strain may carry one or more complementable chromosomal deletions or mutations that are complemented by one or more plasmids.

Also described is a method of producing an Escherichia coli cell carrying recombinant plasmids capable of expressing one or more colonization factors (CFs) associated with enterotoxigenic Escherichia coli bacteria (ETEC) in an increased amount compared to the CFs expressed by ETEC wild-type reference strains. The method comprises the steps of:
assembling in a plasmid genes required for expression of ETEC CFs;
a powerful non-ETEC promoter that controls the expression of the CFs;
a selection marker for plasmid maintenance;
an origin of replication for the plasmid; and
optionally, a means regulating the level of expression of the CFs.

Further described is a vaccine composition against diarrhea comprising at least one Escherichia coli strain according to the invention, together with pharmaceutically acceptable excipients, buffers, and/or diluents, such as excipients, buffers, and/or diluents suitable for oral delivery of the vaccine.

Suitable excipients, buffers, and diluents are known in the prior art, and guidance for appropriate selection can be found in the U.S. or European Pharmacopoeia.

Several types of CFs associated with human pathogenic strains of ETEC, but CFA/I, CFA/II and CFA/IV are the major types currently associated with approximately 40 to 80% of clinical isolates. CFA/I is a single fimbrial antigen, whereas CFA/II and CFA/IV may be composed of more than one type of CF/CS proteins.

CF expression in wild-type ETEC appears to be restricted so that native strains only express a maximum of two or three types of CF antigens and then in certain combinations. Thus, native CFA/II ETEC strains generally express either CS1 together with CS3, CS2 with CS3 or CS3 alone. Similarly, native CFA/IV ETEC strains generally express CS4 with CS6, CS5 with CS6 or CS6 alone. However, e.g., CS1 and CS2 have not been found in the same wild-type strain, and similarly CS4 and CS5 are not expressed together in naturally occurring strains. Furthermore, expression of CS4, CS5 or CS6 together with CS1 or CS2 or CS3, has not been described for wild-type strains.

A minimum requirement that has been proposed for a vaccine against ETEC is that it should have the potential to induce protection against ETEC strains expressing CFA/I and the different subcomponents of CFA/II and CFA/IV, i.e., CS1-CS6. Thus, ETEC vaccines based on wild-type strains may require a minimum of at least five bacterial strains, expressing CFA/I, CS1+CS3, CS2+CS3, CS4+CS6, and CS5+CS6. However, the invention describes a method for producing bacterial cells that are not so restricted in their CF antigen expression, and that also, as a major additional feature, express the relevant CFs at higher levels than found in wild-type strains. Accordingly, the invention provides bacterial cells that over-express ETEC CFs, particularly over-express at least two different CFs each, such as CFA/I+CS2, CFA/I+CS6, CS2+CS4.

Bacterial cells generated according to the invention can be used to manufacture a vaccine against ETEC disease. Thus, further provided is a method to produce a vaccine that is expected to be protective against diarrhea caused by infection with ETEC also in young children. Since the described methods avoid the previous limitations of CF antigen expression in certain naturally occurring combinations, the invention may provide a vaccine against diarrhea comprising as few as three to four bacterial strains, which together over-express CFA/I, CS1, CS2, CS3, CS4, CS5 and CS6, i.e., two CFs by each strain. Thus, the vaccine will, in total, comprise fewer strains, perhaps with the added advantage of being able to use lower doses of each strain than in earlier tested killed ETEC vaccines. Additionally provided is a method of vaccinating a mammal against ETEC diarrhea that involves oral administration of the described cells or vaccines.

The disclosure is particularly directed to a bacterial strain that has been genetically engineered to express from plasmids one or more colonization factors (CFs) associated with enterotoxigenic Escherichia coli bacteria (ETEC) in an amount exceeding the amount of the CFs expressed by the so far characterized ETEC wild-type strains, i.e., in an increased amount compared to the CFs expressed by ETEC wild-type reference strains.

A presently preferred bacterial strain belongs to the family Enterobacteriacae, and is most preferably an E. coli strain.

The expressed CFs contemplated for the purpose of vaccine production according to the invention are associated with ETEC causing intestinal infection and disease in mammals, especially humans.

Preferably, the strains according to the invention express the CFs on the bacterial surface.

A particularly preferred strain is one expressing CFs and carrying one or more plasmids containing:
the genes required for expression and assembly of ETEC CFs;
a powerful non-ETEC promoter that controls the expression of the CFs;
a selection marker for plasmid maintenance;
an origin of replication for the plasmid; and
optionally, a means to regulate the level of expression of the CFs (FIG. 1).

The expression level obtained with the invention of CFs on the surface of bacteria can be detected by an immunological method, e.g., by applying an inhibition ELISA assay, the level of expression of the CFs is at least three-fold higher or, by applying a dot blot test, is at least five-fold higher, than on any to date characterized ETEC wild-type strain expressing the corresponding CF.

In certain embodiments, the strain in question is a strain that does not express an antibiotic resistance gene.

In certain embodiments, the strain of the invention carries a complementable chromosomal deletion or mutation.

The plasmids used in a strain of the invention may be one that complements a chromosomal mutation.

In certain embodiments, the CFs that are expressed by a strain of the invention are expressed in a form that allow them to react with specific antibodies raised against corresponding CFs from ETEC strains originally isolated from the stool of a mammal with intestinal ETEC infection.

In certain embodiments, the CFs that are expressed by a strain expressed in a form such that when the strain is used in an effective amount for immunization of a mammal, leads to formation of antibodies against the expressed CFs that can react with corresponding CFs from ETEC strains originally isolated from the stool of a mammal with intestinal ETEC infection.

In certain embodiments, the CFs that are expressed by a strain expressed in a form that after inactivation of the strain by formalin treatment or other means, allows them to react with specific antibodies raised against corresponding CFs from ETEC strains originally isolated from the stool of a mammal with intestinal ETEC infection.

In certain embodiments, the CFs that are expressed by a strain of the invention are expressed in a form that after inactivation of the strain by formalin treatment or other means, when the strain is used in an effective amount for immunization of a mammal, leads to formation of antibodies against the expressed CFs that can react with corresponding CFs from ETEC strains originally isolated from the stool of a mammal with intestinal ETEC infection.

Examples of CFs for the purpose of the invention comprise CFA/I, CS1, CS2, CS3, (CS4), CS5 or CS6, alone or in any combination of two or more of the CFs expressed from the same or different plasmids in the same or different bacterial cells. Advantageously, unnatural combinations of two or more of the CFs are co-expressed from the same bacterial cell.

Bacterial strains may be cultured by methods for in vitro culturing of the strains in liquid media providing high-level surface expression of the CFs.

A cultured strain may be inactivated by using mild treatment with formalin or phenol or other means, thereby preventing the strain from replication, and resulting in a strain that retains the over-expressed CFs in essentially the same amounts (at least 50% of the original amount), and with essentially the same reactivity with antibodies and almost the same immunogenicity as for the strain before the inactivation.

A bacterial strain according to the invention or a combination of such strains are suitable for use in a method of vaccination against diarrhea, or for use in the manufacture of a vaccine.

One or several of the bacterial strains is (are) especially suitable for use in a method of vaccinating a mammal against diarrhea, which comprises administering to the mammal a strain or combination of strains according to the invention.

In certain embodiments, one or several of the bacterial strains of the invention is (are) used alone or in combination as a vaccine, for vaccination of a mammal, such as a piglet, a calf, a lamb or a horse, or, in particular, a human being. Such a vaccine is preferably administered by the oral route.

### DESCRIPTION OF THE DRAWINGS

FIG. 1. The figure shows typical features of an over-expression plasmid as outlined in Example 1. Cfa-A/B/C/E are genes required for expression and assembly of CFA/I. Expression of genes is under the control of the tac promoter. The plasmid is maintained using ampicillin as a selection marker expressed by bla, and the origin of replication is derived from ColEI (pBR322). Expression of CFA/I is controlled by the lac repressor expressed by lacIq.

FIG. 2. The figure shows typical features of over-expression plasmids as described for FIG. 1. Additionally, the plasmid pAF-thyA-CFA/I-AMP (FIG. 2, top) contains a gene, thyA, encodes the production of thymine to confer thymine independence on an otherwise thymine-dependent strain. The plasmid pAF-thyA-CFA/I (FIG. 2, bottom) has the same features as pAF-thyA-CFA/I-AMP, however, lacks the selection marker for ampicillin as expressed by bla and, therefore, this plasmid is maintained using thymine as a selection marker expressed by thyA.

FIG. 3. The figure shows construction of pAF1-CS2 expression vector. The entire CS2 operon and the flanking regions were amplified and cloned into the expression vector pAF1, creating pAF1CS2, as described in results (1B).

FIG. 4. The figure shows inhibition ELISA titers of TOP10-pAF1-CS2 and reference 58R957 strains. The expression level of CS2 was determined by inhibition ELISA, as described in materials and methods. The bars indicate the geometric mean of ELISA titers (+SD) of three preparations (triplicates). Asterisks indicate significant difference (P<0.05) between CS2 expression by TOP10-pAF1-CS2 and the reference strain 58R957, as determined by Student's t-test.

FIG. 5. The figure shows an electron micrograph of immunogold-labeled TOP10-pAF1-CS2 strain using a monoclonal antibody against CS2 (MAb anti-CS2 10:3).

FIG. 6. The figure shows construction of pMT-CS2 expression vector, as described in Example 5.

FIG. 7. The figure shows the level of CS2 expression on TOP10-pMT-CS2 (lane 2) and reference 58R957 (lane 1) strains. Immunological reactivity of anti-CS2-specific monoclonal antibody (10:3) with each strain was demonstrated by dot blot. Each strain was cultured in LB, and the initial concentration of the bacteria was 10⁹/ml. The dilutions are indicated alongside the strip.

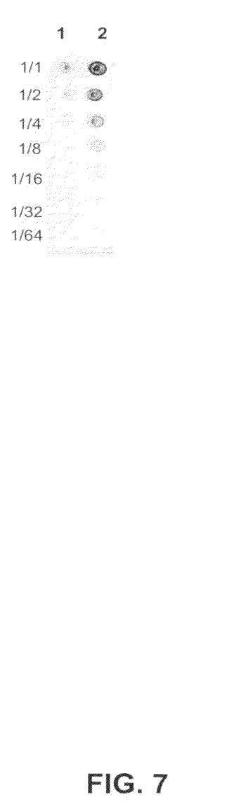

FIG. 8. The figure shows electron micrographs of immunogold-labeled of TOP10-CFA/I-CS2 strain using monoclonal antibodies against CFA/I (MAb 1:16) (top) and CS2 (MAb 10:3) (bottom).

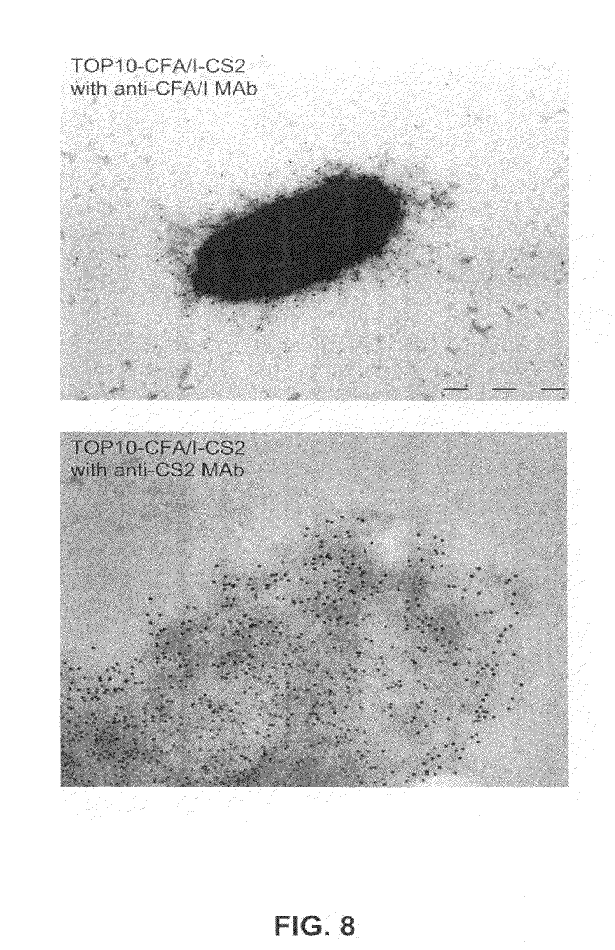

FIG. 9. The figure shows the level of CFA/I and CS2 expression on killed TOP10-CFA/I-CS2 and the corresponding reference strains 325542-3 (CFA/I) and 58R957 (CS2). Immunological reactivity of anti-CFA/I and anti-CS2 specific monoclonal antibody with each strain was demonstrated by dot blot. Each strain was cultured in LB, and the initial amount of the formalin killed bacteria was 10⁹/ml. The dilutions are indicated alongside the strip.

FIG. 10. The figure shows IgA antibody titers in serum following oral immunization of mice with killed TOP10-CFA/I-CS2 and the reference strains 325542-3 (CFA/I) and 58R957 (CS2). Serum samples were collected two weeks after the second immunization, and the anti-CFA/I and anti-CS2 IgA titers were determined by ELISA. Titers are shown as reciprocal geometric mean titers (+SE) of three animals in each group, immunized with TOP10-CFA/I-CS2 or the reference strains.

FIG. 11. The figure shows IgG+M antibody titers in serum following oral immunization of mice with formalin killed TOP10-CFA/I-CS2 and the reference strains 325542-3 (CFA/I) and 58R957 (CS2). Serum samples were collected two weeks after the second immunization, and the titers of anti-CFA/I and anti-CS2 IgG+M titers were determined by ELISA. Titers are shown as reciprocal Log₁₀ geometric mean titers (+standard error) of three animals in each group, immunized with TOP10-CFA/I-CS2 or the reference strains. Asterisk indicates significant difference (P<0.05), compared to the reference strain, as determined by student's t-test.

FIG. 12. The figure shows antibody titers in serum following oral immunization of mice with killed TOP10-CFA/I-052 and the reference strains 325542-3 and 58R957. Serum samples were collected two weeks after the second immunization and the titers of anti-CT IgA was determined by ELISA. Titers are shown as reciprocal Log₁₀ geometric mean titers (+standard error) of three animals in each group, immunized with TOP10-CFA/I-CS2 or the reference strains.

### DETAILED DESCRIPTION OF THE INVENTION

### Materials and Methods

### Bacterial Strains and Culture Conditions

Strains described in this application are listed in Table 1. Construction of recombinant strains over-expressing CFs is exemplified by construction of a strain over-expressing CFA/I in Example 1.

Strains were kept frozen at −70° C. in a glycerol-containing freezing medium until used. After inoculation of an agar plate at 37° C. over night to ascertain growth and purity, bacteria were grown in CFA broth (Casamino acids 10 g, yeast extracts 1.5 g, MgSO₄7H₂O 102 mg, MnCl₂4H₂O 8 mg per liter), at 37° C. with shaking for 16 to 18 hours.

### Over-Expression of Recombinant CFs on the Bacterial Surface

Overnight cultures of the recombinant CF-expressing strains were diluted 1/100 in CFA broth supplemented when necessary with 100 μg/ml of ampicillin. Resulting cultures were incubated for two hours at 37° C. with shaking (150 revolutions/minute in a rotary shaker). To induce expression of the CFs on the bacterial surface, isopropyl-β-D-thiogalactopyranoside (IPTG) was then added to a final concentration of 1 mM, and incubation continued under the same conditions for another four hours. The bacteria were then harvested and re-suspended in PBS.

Isolation of thyA E. coli K12 Mutant

Construction of a thyA (thymidilate synthetase) mutant of the E. coli K12 (TOP10) strain was done using trimethoprim selection as previously described (Stacey and Simson, 1965) except that M9 medium was supplemented with Casamino acids (Difco, Beckton Dickinson, Sweden) to a final concentration of 0.05% and trimethoprim was used at a concentration of 200 μg/ml.

### Inactivation of Bacteria

To inactivate (kill) the bacteria, cultures of each strain were washed and re-suspended in PBS to a density of ˜10¹⁰ bacteria/ml. The inactivating agent, e.g., formalin, was added to an appropriate final concentration, i.e., 0.05 to 0.2 M, and the suspension was incubated for two hours at 37° C. under gentle agitation, after which it was left to stand at 4° C. for three days without agitation. After washing and resuspension with the same volume of PBS, 100 μl of the bacterial suspension was spread onto blood agar plates and incubated at 37° C. for up to a week to check for growth, before the bacteria were analyzed for the amount of the respective CF on the bacterial surface.

### Methods to Evaluate the Level of Surface CFs on Recombinant and Reference Strains

Two different methods, the dot blot and the inhibition ELISA assays, both of which allow determination of surface-expressed CFs, were applied for evaluating the amount of each CF on the recombinant bacteria and for comparison with the corresponding reference strains:

a) Dot Blot

To evaluate the presence and amount of CF on each recombinant and reference strain, respectively, individual dot blot assays were performed using a monoclonal antibody (MAb) against the corresponding CF and corresponding reference strains. The MAbs used in the different tests are listed in Table 2. The test was carried out as follows:

A nitrocellulose membrane (Sorbent AB, Sweden) was soaked in phosphate-buffered saline (PBS) and allowed to air dry. Thereafter, 2 μl aliquots of two- or three-fold dilutions of whole bacteria in PBS (10⁹ bacteria/ml; OD₆₀₀=0.8) were applied to the membrane and air dried again. Blocking was performed with 1% (wt/vol.) bovine serum albumin (BSA) (Sigma-Aldrich) in PBS for 30 minutes with gentle agitation. All incubations were performed at room temperature. After washing twice in PBS, the nitrocellulose membrane was incubated with corresponding anti-CF MAb (appropriately diluted in 0.1% BSA-PBS-0.05% TWEEN® 20 (Sigma-Aldrich)) overnight with gentle agitation. The membrane was washed thrice in PBS-0.05% TWEEN®, and an anti-mouse IgG-horseradish peroxidase conjugate (Jackson ImmunoResearch; GTF Sweden), diluted in 0.1% BSA-PBS-0.05% TWEEN®, was added. After incubation for two hours, the membrane was washed twice in PBS-TWEEN® and once in PBS and was then developed for five to ten minutes by using hydrogen peroxide (0.012%) as the substrate and 4-chloro-naphthol (Bio-Rad Laboratories) as the chromogen. Stained dots on a white background indicated positive results. The highest reciprocal dilution of a 10⁹ bacteria/ml suspension causing a visible dot on the nitrocellulose sheet was determined for each strain.

b) Inhibition ELISA

To determine the amount of CFs on the surface of the recombinant and reference strains, respectively, the capacity of the CF-expressing bacteria to inhibit binding of corresponding anti-CF MAb to the homologous solid-phase-bound CF was assayed by an ELISA inhibition method. The MAbs and the CFs used for coating in the different tests are listed in Table 2. Bacteria were tested in an initial concentration of 10⁹ bacteria/ml and the test was carried out as described below in Inhibition ELISA for quantification of ETEC CFs/CS-factors. The highest interpolated reciprocal dilution of the bacterial suspension tested causing 50% inhibition of the Mab to the corresponding solid phase antigen was determined.

### Animal Immunizations

Female Balb/c mice (six to eight weeks of age) were used for all immunization experiments. Inactivated bacteria of the recombinant and reference CF-expressing strains, respectively, were given in a dose of 10⁹ bacteria, together with 10 μg CT orally under anesthesia as previously described (Rhagavan et al., 2002). All animals were given two identical immunizations two weeks apart, and blood for preparation of sera was collected immediately before the first dose and two weeks after the second dose.

### ELISA for Determination of Anti-CF Antibody Titers

Antibody titers of IgA and IgG isotypes against the appropriate CF antigen were determined in pre- and post-immunization sera of the mice by ELISA. ELISA microtiter plates were coated with 1 μg/ml of purified CF at 37° C. overnight (100 μl per well). After the plates were blocked with a 1% BSA-PBS solution at 37° C. for 30 minutes, three-fold dilutions of sera in PBS containing 0.1% BSA-TWEEN® were incubated in the plates at room temperature for 1.5 hours. Bound antibody was then demonstrated by the addition of anti-mouse HRP-conjugated anti-IgA or anti-IgG+M conjugate using H₂O₂ as enzyme substrate and OPD as chromogen. Titers were determined as the reciprocal dilution giving an absorbance of 0.4 at 450 nm above background after reacting the enzyme with its substrate for five to twenty minutes. Sera from different animals were analyzed individually and the geometric means and standard errors of the means (SE) of reciprocal individual titers in each group were calculated.

### Detailed Description of Inhibition ELISA for Quantification of ETEC CFs/CS-Factors

Determination of the amount of CFs on the surface of whole (live or inactivated) bacteria by inhibition ELISA is performed as follows:

A. Coat microtiter plates (e.g., NUNC polystyrene ELISA plates) with the respective purified CF antigen diluted in phosphate-buffered saline (PBS), 100 μl/well, at 37° C. overnight. Suitable antigens and coating concentrations are shown in Table 2. Store plates at 4° C. for up to 14 days until use.

B. Block uncoated plates with 1% bovine serum albumin (BSA), 200 μl/well, 37° C. for 30 minutes.

C. Add 60 μl/well of 0.1% BSA-PBS containing 0.05% TWEEN® 20 to all wells of the uncoated, blocked plate except the first well in a row. Add 90 μl of sample to the first well in the rows. Mix and transfer 30 μl from this well to the second well and continue this three-fold dilution in another six wells; leave only BSA-PBS-TWEEN® in the last well of each row.

D. Add 60 μl/well of corresponding monoclonal antibody (MAb) diluted in 0.1% BSA-PBS-TWEEN®. Designation, isotype and suitable concentration of the respective MAb are shown in Table 2.

E. Incubate the plate at room temperature with gentle shaking for one hour.

F. Wash the CF antigen-coated plate twice with PBS.

G. Block remaining binding sites of the solid phase of the CF antigen-coated plate by incubating the plate with 1% BSA, 200 μl/well, at 37° C. for 30 minutes.

H. Wash the plate once with PBS-TWEEN®. Transfer 100 μl/well of each mixture from the non-coated “inhibition-plate” starting with the last well (containing BSA-PBS-TWEEN®) to a CF antigen-coated, BSA-blocked plate.

I. Incubate at room temperature for 90 minutes with the plate standing still.

J. Wash the plate three times with PBS-TWEEN®.

K. Add anti-mouse Ig-horse radish peroxidase (HRP) conjugate diluted in 0.1% BSA-PBS-TWEEN®, 100 μl/well. (Suitable conjugate concentration has to be determined for each new conjugate lot.) Incubate at room temperature for 60 to 90 minutes.

L. Wash the plate three times with PBS-TWEEN®.

M. Add the enzyme substrate, i.e., ortophenylene diamine (OPD) prepared by dissolving 10 mg of OPD in 10 ml of 0.1 M sodium citrate buffer (pH 4.5) to which 4 μl of 30% hydrogen peroxide has been added immediately before use. Add 100 μl of this mixture to each well and read after ten or twenty minutes at 450 nm in a spectrophotometer (e.g., Labsystem, Multiskan). The absorbance in the last well (buffer+MAb) should be ˜1.0.

N. Make curves by plotting absorbance values versus dilution of the sample, and determine 50% inhibitory concentration of each specimen (for calculations see below).

### Determination of 50% Inhibitory Titer

Maximum Absorbance: MAb+BSA-PBS-TWEEN® (mean of absorbance values in the last well of each row)

Minimum Absorbance: MAb+highest concentration of corresponding CFA reference

$$50 \% inhibition concentration = \frac{Max Abs - Min Abs}{2} + Min Abs$$

The 50% inhibitory titer is determined as the reciprocal dilution of bacteria causing 50% inhibition of the absorbance when reacting the MAb with the corresponding solid phase bound CF antigen.

The references cited herein are hereby incorporated in this specification by this reference.

### EXAMPLES

### Example 1

### Construction of a Recombinant Strain Over-Expressing CFA/I

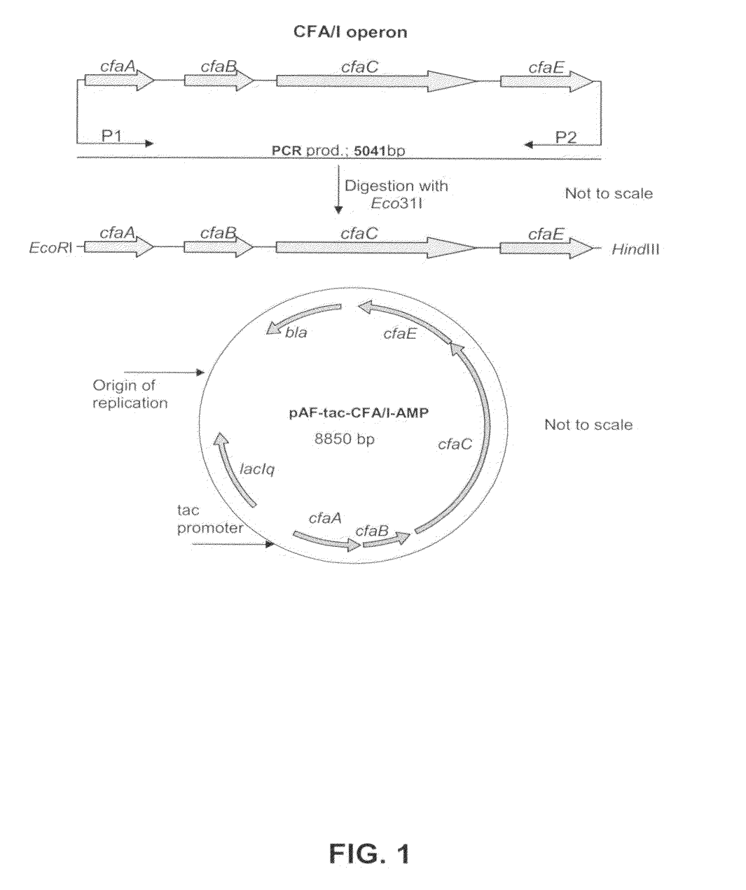

The construction of E. coli strains with the capacity to over-express ETEC CF antigens on the bacterial surface is here exemplified with the description of the generation of such a strain, which when grown in vitro under appropriate cultivation conditions, can produce an excessive quantity of E. coli colonization factor antigen I (CFA/I). The approach taken was to clone the entire CFA/I operon, consisting of four genes, from a CFA/I producing wild-type ETEC strain into a plasmid expression vector, and then to introduce this plasmid into the E. coli K12 (E. coli TOP10) strain. This was accomplished by standard procedures for DNA manipulation, which were described by Sambrook and Russell (2001) or according to instructions supplied with reagents. PCR was used to amplify the relevant genes of the CFA/I operon from the CFA/I positive wild-type reference strain 325542-3. Template DNA was prepared by taking a fresh colony of bacteria from an agar plate and suspending the cells in 100 μl double-distilled sterile water. The suspension was boiled in a water bath for five minutes and subsequently spun at full speed in a bench top centrifuge for five minutes. Aliquots of the resulting supernatant were used as template. Amplification was carried out using appropriate forward and reverse primers (shown in Table 1) and the Expand High Fidelity PCR System (Roche Diagnostics GmbH, Germany). CFA/I-forward is homologous to a sequence 22 base pairs (bp) upstream of cfaA and carries restriction sites for EcoRI and Eco311, whereas CFA/I-reverse, which extends 1 by downstream the cfaE, carries restriction sites for HindIII and Eco311, at the 5′ end (FIG. 1). PCR conditions were as follows: 95° C. for five minutes, 31 cycles of 94° C. for 15 seconds, 58° C. for 30 seconds and 68° C. for four minutes, with a final extension of seven minutes at 72° C. The resulting 5041 bp fragment carries the cfaA, cfB, cfaC, and cfaE genes (CFA/I) necessary for production and assembly of CFA/I fimbriae. The amplified CFA/I DNA was then restricted with Eco311, resulting in a fragment flanked with EcoRI and HindIII compatible ends (FIG. 1). Following restriction of the expression vector pAF-tac (Sadeghi et al., 2002) with EcoRI and HindIII, the digested PCR fragment and the vector were mixed and ligated using T4 DNA ligase. Ligated DNA was electroporated into the E. coli K12 strain TOP10 (Invitrogen, Life Technologies, Sweden). Colonies were initially screened for the presence of CFA/I DNA by PCR and positive clones were further analyzed by restriction analysis of isolated plasmids. It was found that out of 44 tested colonies, 34 contained the plasmid harboring CFA/I. The 8850 bp plasmid in which the CFA/I operon is located downstream of the IPTG-induced tac promoter was named pAF-CFA/I-AMP. One colony was then selected and used as parental colony of recombinant E. coli K12 strain carrying pAF-CFA/I-AMP (TOP10-CFA/I). The surface over-expression of CFA/I by this strain was then examined as described in Example 2.

A similar approach was also applied to produce recombinant strains over-expressing CS2, CS4 and CS6, respectively. The primers (forward and reverse) used for construction of these recombinant strains are listed in Table 1.

### Example 2

### Demonstration of Over-Expression of CFs on the Surface of Recombinant E. coli K12 Strains

To determine the expression of different ETEC CFs on recombinant E. coli K12 strains, two different assays, i.e., dot blot and inhibition ELISA, were used. In the dot blot assay, 2 μl of two- or three-fold dilutions of whole bacteria in PBS in initial concentration of 10⁹ bacteria/ml, were applied to a nitrocellulose membrane. Following incubation of the membrane with corresponding anti-CF MAb, the membrane was washed, incubated with anti-mouse IgG-horseradish peroxidase conjugate, and then developed as described in Materials and Methods. Expression of CFs on the bacterial surface of recombinant and reference strains was also determined by use of inhibition ELISA, in which the capacity of the CF on the respective strain to inhibit binding of corresponding anti-CF MAbs to solid-phase-bound CF is determined, as described in Inhibition ELISA for quantification of ETEC CFs/CS-factors.

The results of testing the capacity of the recombinant E. coli K12 strains (E. coli TOP10) to express ETEC CF as compared to the reference strain, i.e., clinical strains that have previously been found to express the highest levels of respective ETEC CFs, in the dot blot test are shown in Table 3. The titers shown represent the mean highest reciprocal dilution of each strain giving a visible dot on the nitrocellulose membrane. Asterisks indicate significant difference (P<0.01) when comparing the level of CFs between each recombinant strain and the corresponding reference strain, as determined by student's t-test. As shown in Table 3, all the recombinant strains express considerably higher levels of CF than the corresponding reference strains.

The capacity of E. coli K12 recombinant strains to express ETEC CFs, as compared to the reference strains as determined by inhibition ELISA, is shown in Table 4. The titers indicate the level of the ETEC CFs on each tested strain. Asterisks indicate a significant difference (P<0.05) when comparing the level of CFs between each recombinant strain and the corresponding reference strain, as determined by student's t-test. As shown, the recombinant strains express considerably higher levels of CFs than the reference strains.

### Example 3

### Introduction of a Non-Antibiotic Resistance Marker in the Recombinant CFA/I Over-Expressing E. coli K12 Strain (TOP 10-CFA/I Strain)

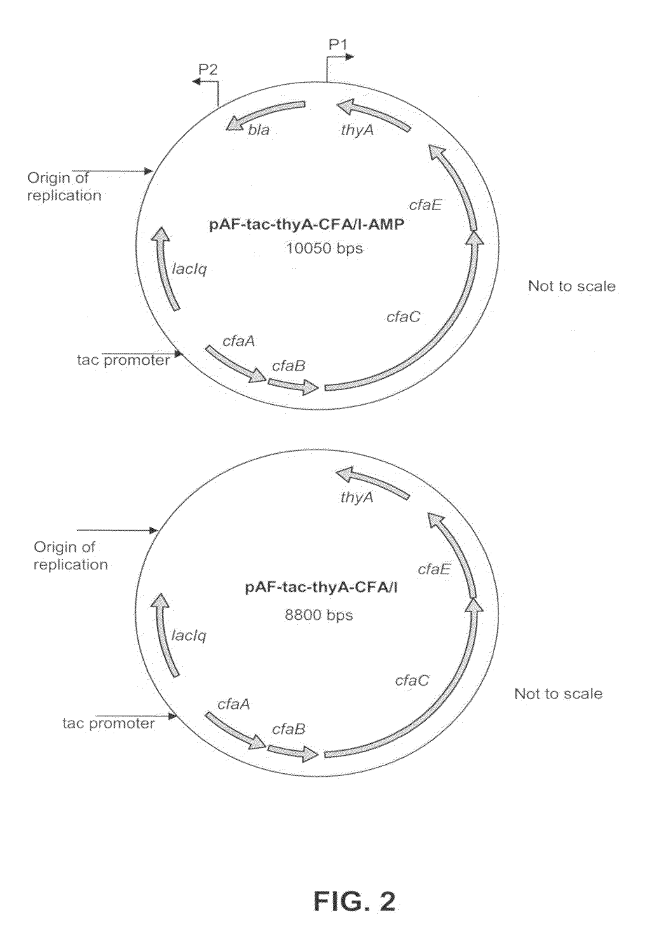

A selection marker, like an antibiotic resistance marker, is needed for the maintenance of expression vectors in recombinant strains. However, to eliminate the possibility of antibiotic residues in vaccine preparations and to prevent the possibility of horizontal spread of genes encoding antibiotic resistance in the environment, such markers should be avoided in vaccine strains. We therefore replaced the β-lactamase gene (bla) present in pAF-thyA-CFA/I-AMP with a non-antibiotic marker thyA (which complements a thyA mutation in the host and confers thymine independence on an otherwise thymine-dependent strain) in the over-expressing TOP10-CFA/I recombinant strain. A 1200 bp fragment carrying the thyA gene flanked by SalI and XhoI restriction sites was ligated into pAF-tac-CFA/I-AMP digested with XhoI (FIG. 2). This resulted in the 10050 bp plasmid pAF-tac-thyA-CFA/I-AMP, which was isolated by electroporation of thyA⁻ derivative of E. coli K12 strain and selecting for thymine independence and ampicillin resistance. The orientation of the thyA gene in the pAF-tac-thyA-CFA/I-AMP was confirmed by DNA sequencing and the primers P1 and P2 (Table 1) were designed in order to remove the ampicillin resistance gene by reverse PCR. PCR amplification resulted in an 8800 bp fragment carrying the entire plasmid except for the ampicillin resistance gene. This was then restricted with Eco311 and self ligated. The resulting plasmid was pAF-tac-thyA-CFA/I. CFA/I expression was confirmed in the resulting strain by dot blot and inhibition ELISA following induction of cultures with IPTG. As shown in Table 5, no significant difference was observed when the expression level of CFA/I was compared between the thyA-complemented recombinant E. coli K12 strain (TOP10), expressing CFA/I from pAF-tac-thyA-CFA/I, and the original recombinant E. coli K12 strain (TOP10) carrying the pAF-tac-CFA/I-AMP.

### Example 4

### A Method for Inactivating CFs of Over-Expressing Recombinant E. coli Strains, with Retention of Antigenic and Immunogenic Properties

For the immunization experiments, the recombinant TOP10-CFA/I strain and the reference strain were inactivated to prevent multiplication in the intestine after oral administration. To accomplish inactivation of the bacteria, both the recombinant and the reference strains cultures were inactivated by mild formalin treatment. This was done by agitating each bacterial suspension (10¹⁰ bacteria/ml) gently with formalin for two hours at 37° C. followed by at 4° C. for three days without agitation, as described in Materials and Methods. To verify that the bacteria had been inactivated (killed) by this procedure, 100 μl volumes of the treated bacterial suspensions were spread onto blood agar plates and incubated at 37° C. for a week to check for growth. For both strains tested, no colonies were found after reading the plates daily for up to a week after inoculation, verifying the killing effect of the formalin treatment.

To evaluate the CFA/I antigenicity after formalin treatment, the level of CFA/I expression on the recombinant E. coli TOP10-CFA/I, as well as on the reference strain was tested. As shown in Table 6, no significant difference was found in the level of CFA/I expression for both strains before and after formalin treatment.

To evaluate the immunogenicity of the formalin-inactivated TOP10-CFA/I strain as compared to the reference strain, mice were orally immunized with corresponding doses of the respective strain, and immune responses compared. All mice were given two doses of the respective strain in a dose of 10⁹ bacteria, together with 10 μg cholera toxin and sera were collected immediately before and then two weeks after the second dose. Sera were analyzed by means of a CFA/I ELISA as previously described (Rudin et al., 1994). As shown in Table 7, the recombinant strain induced higher IgA (significantly) as well as IgG+IgM titers against CFA/I than the reference strain.

### Example 5

### Construction and Characterization of a Strain that Co- and Over-Expresses CFA/I and CS2

In this example, we describe the construction of a strain that co-expresses, in increased amounts, two CF proteins on the same bacterial cells.

### Material and Methods

### Cloning of CS2 Operon

The genes CotA, CotB, CotC and CotD, which are required for expression and assembly of CS2 antigen, were amplified by PCR using the primers CS2-forward and CS2-reverse (Table 8). The amplified fragment was then ligated into pAF, pMt and pBAD vectors, resulting in different E. coli K12 recombinant strains (TOP10) expressing the CS2 antigen.

### Expression of CFA/I and CS2

An overnight culture of TOP10-CS2 and E. coli K12-CFA/I-CS2 were diluted 1/100 in CFA broth, supplemented with 100 or 12.5 μg/ml of ampicillin or chloramphenicol, respectively, and incubated for two hours at 37° C. and 150 revolutions/minute, followed by addition of IPTG to the final concentration of 1 mM and incubation with the same conditions for four hours. The bacteria were then harvested and re-suspended in PBS.

### Dot Blot Test

Specific anti-CFA/I MAb 1:6 and anti-CS2 MAb 10:3 were used to evaluate the expression of CFA/I or CS2, respectively, on the cloned strains, as described (Binsztein et al., 1991). Briefly, 2 μl of bacterial cultures (10⁹ bacteria/ml in PBS) that have been washed once with PBS, and induced with IPTG for expression CFA/I, were applied on the nitrocellulose filter papers and incubated with the MAbs followed by goat anti-mouse IgG, conjugated with HRP, for 1.5 hour each. The final development was performed by 4-chloro-1-naphtol-H₂O₂ in TBS for up to 15 minutes.

### Electron Microscopy

Ten μl of each bacterial suspension (10¹⁰ bacteria/ml in PBS), that had been washed once with PBS, were applied on parafilm. Formvar-coated grids were put on the suspension for two minutes, with the grids placed 15 cm below a lamp to increase the temperature of the sample. The grids were then washed twice, ten seconds each, by applying them on 25 μl of PBS-1% BSA on parafilm, followed by incubating the grids for 15 minutes with 25 μl specific monoclonal antibody diluted in PBS-TWEEN® 0.05%-BSA 0.1%. The grids were washed six times with PBS-1% BSA, as above, and then incubated for 15 minutes with anti-mouse IgG-gold conjugate (Amersham International, Amersham, UK) in PBS-0.1% BSA-0.05% TWEEN®. The grids were then washed three times with PBS-0.1% BSA, and three times with distilled water. Negative staining was performed by applying the grids on 25 μl of 1% ammonium molybdate (pH 7.0) for 50 to 60 seconds, followed by air-drying the grids on a filter paper for five minutes. The grids were stored at 4° C. until examined by electron microscopy.

### Mouse Immunization

Female Balb/c mice (six to eight weeks of age) were used for the immunizations by the oral route. Cultures of formalin killed reference strains 325542-3 and 58R957, and the recombinant CF-induced strain TOP10-CFA/I-CS2, were washed and re-suspended in PBS to the desired bacterial density. 10⁹ bacteria together with 10 μg CT in 0.2 ml PBS were used for each immunization. All mice were given two identical immunizations two weeks apart, and bleedings were collected immediately before the first dose and two weeks after the second dose.

### Statistical Analysis

All ELISA and inhibition ELISA experiments were performed in duplicate and repeated at least three times on different days. Dot blot experiments for each particular test were repeated at least twice. Statistical analyses were conducted by the student's t-test and P<0.05 was regarded significant.

### Results

### Cloning of CS2

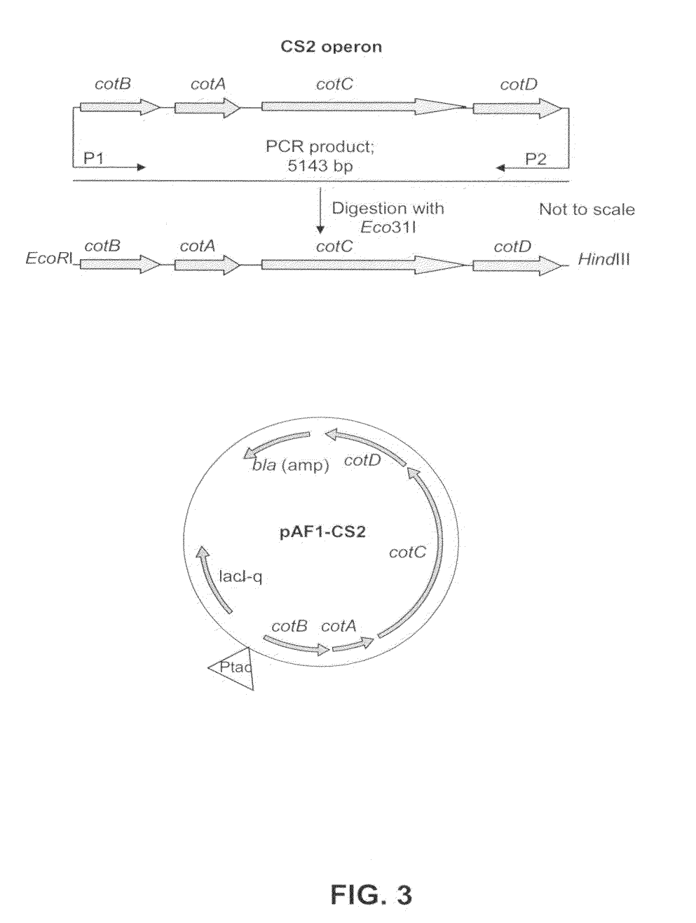

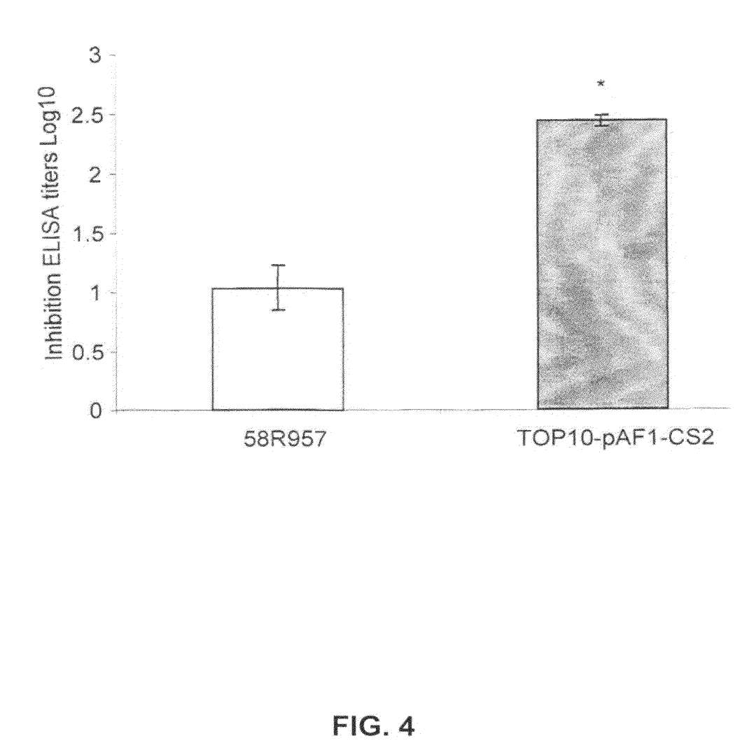

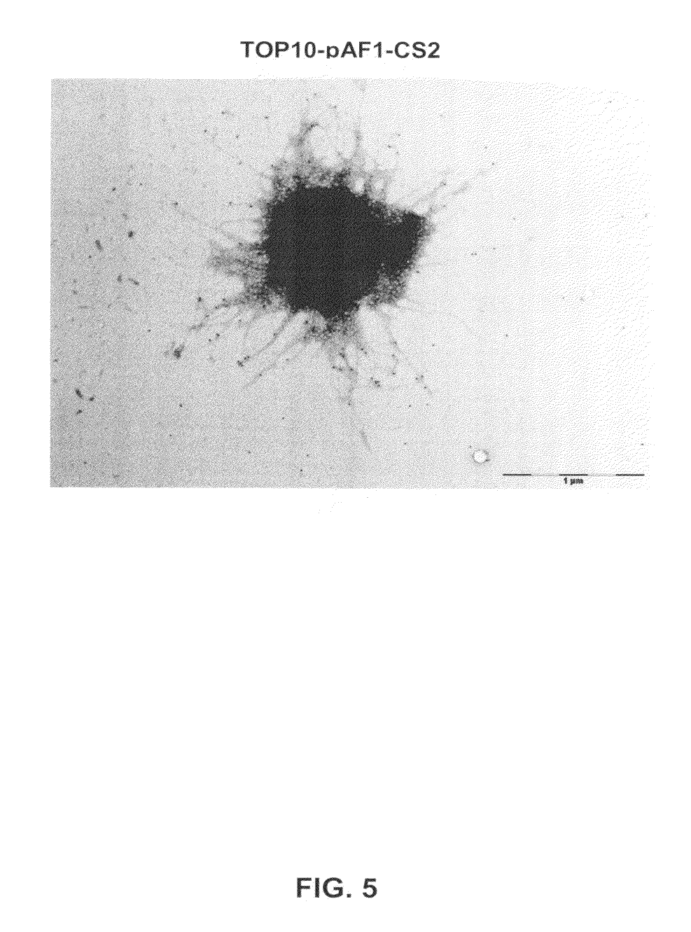

We have described the construction and immunogenicity of a non-toxigenic E. coli strain, TOP10-CFA/I over-expressing the colonization factor I (CFA/I) of ETEC (see example above). The same expression vector, pAF1, and the strategy were applied to first express CS2 on E. coli K12 strain (TOP10), as depicted in FIG. 3. The CS2 operon consists of genes cotA, cotB, cotC, and cotD (GenBank accession number Z47800), which are required for expression and assembly of CS2. The entire operon was amplified by PCR, which resulted in a 5143 bp (FIG. 3). The fragment was then digested with Eco311, cloned into the pAF1 vector, resulting in pAF1-CS2 (FIG. 3), followed by electroporation of the E. coli K12 strain (TOP10), which resulted in TOP10-pAF1-CS2. By applying inhibition ELISA assay, we found that the strain TOP10-pAF1-CS2 expressed approximately twenty-fold more CS2 than the reference strain (58R957) (FIG. 4). To demonstrate the presence of the CS2 on TOP10-pAF1-CS2 strain, IEM was performed using the specific anti-CS2 MAb 10:3 as antibody. The TOP10-pAF1-CS2 strain expressed large amounts of CS2 antigens on its surface with gold particles along the whole CF antigens (FIG. 5).

### Co-Expression of CS2 and CFA/I

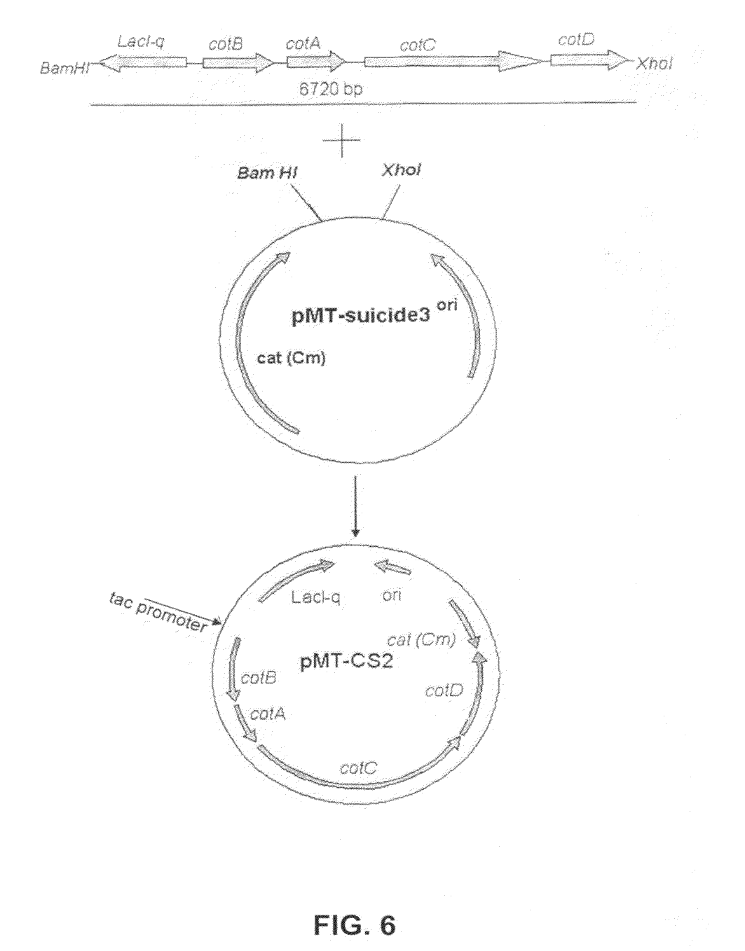

In an attempt to co-express both the CS2 with CFA/I colonization factors, we first cloned the CS2 operon into another vector, pMT, harboring resistance marker for chloramphenicol (Cm, cat), as depicted in FIG. 4. The pAF1-CS2 vector was first cut with XhoI and BamHI restriction enzymes, resulting in a 6720 bp fragment, which was cloned onto the pMT vector already cut with XhoI and BamHI (FIG. 6). The resulting expression vector, pMT-CS2, was then propagated in an E. coli K12 strain (TOP10), resulting in TOP10-pMT-CS2. By applying dot blot assay, we found that TOP10-pMT-CS2 strain expresses eight-fold more CS2 than the reference strain 58R957 (data not shown). We then electroporated both pAF-tac-CFA/I-Amp and pMT-tac-CS2-Cm expression vectors into TOP10 strain, resulting in TOP10-CFA/I-CS2 strain. Inhibition ELISA assay showed that the TOP10-CFA/I-CS2 strain expresses several-fold more CFA/I and CS2, respectively, compared with the respective reference strains (Table 10). Culturing the clone, and examining the expression of both surface antigens at different times by using dot blot, showed stable expression of both CFA/I and CS2 on TOP10-CFA/I-CS2.

TOP10-CFA/I-CS2 and the reference strains 325542-3 and 58R957 were examined for CF antigen structures by IEM, using specific monoclonal antibodies anti-CFA/11:6 and anti-CS2 10:3, respectively. Under identical growth conditions, the TOP10-CFA/I-CS2 strain expressed considerably longer and higher amounts of the CF antigens on its surface, and also considerably more gold particles along the whole CF antigens, compared to the reference strain.

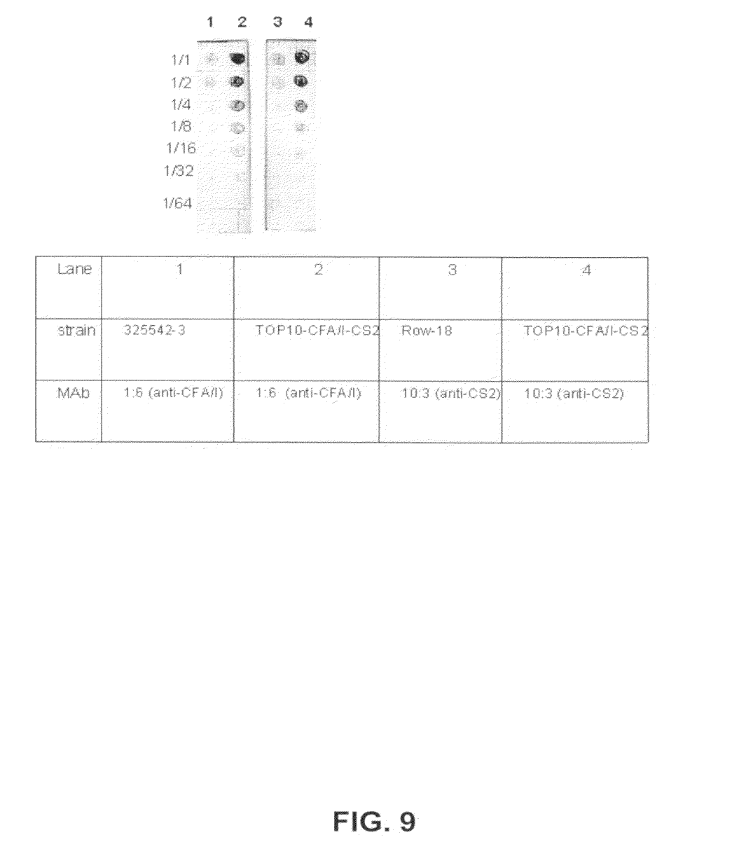

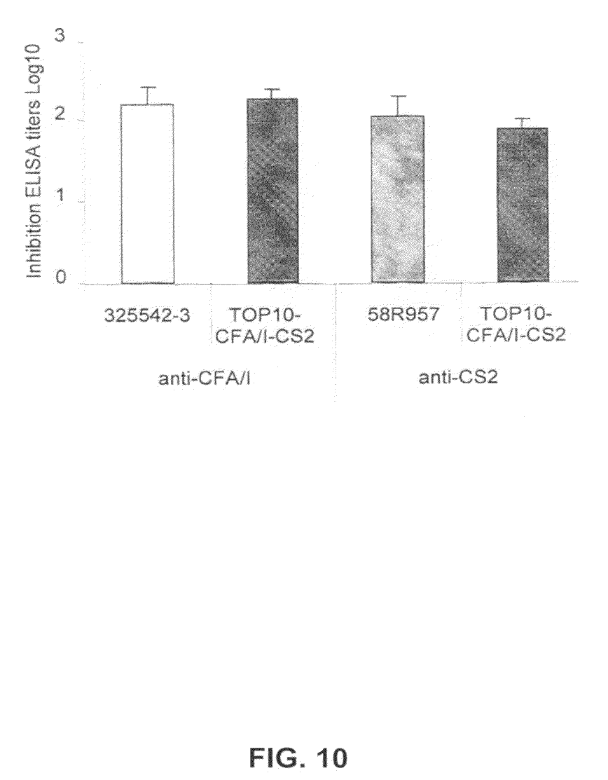

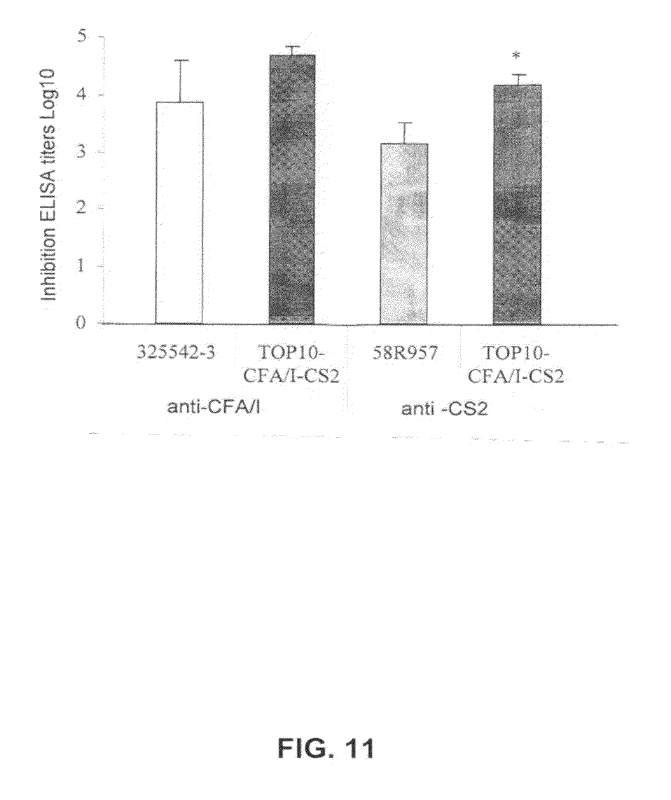

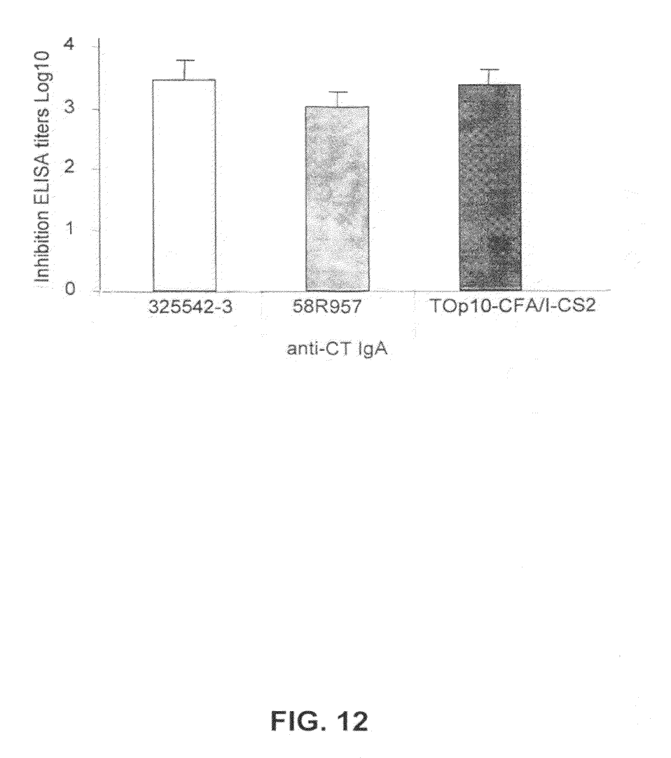

In order to examine the immunogenicity of TOP10-CFA/I-CS2 strain in mice, the cloned strain was killed by formalin as described in Materials and Methods. Following the formalin treatment, the expression level of CFA/I and CS2 was examined by dot blot, which showed that the cloned strain still expresses significantly higher amounts of both CFA/I and CS2, compared to each of the respective reference strains (FIG. 9). The immune responses induced by TOP10-CFA/I-CS2 were compared to those induced by each of the reference strains 325542-3 and 58R957. Balb/c mice were immunized with two identical oral administrations of formalin killed bacteria of the respective strains. Following the immunization, the sera of mice were assayed for antibodies against each of the CF antigens by ELISA, which showed that all animals immunized with TOP10-CFA/I-CS2 or each of the reference strains responded with high levels of both serum IgA and IgG+M (FIGS. 10 and 11). The IgA levels in sera of mice immunized with TOP10-CFA/I-CS2 against CFA/I and CS2 were comparable with those of mice immunized with each of respective reference strains, although still indicating the immunogenicity of the cloned strain (FIG. 10). Although no significant difference in IgG+M level against CFA/I of the cloned and the reference strain 325542-3 was observed, the level of IgG+M against CS2 in sera of mice immunized with the cloned strain was significantly higher compared with that of the reference strain 58R957 (FIG. 11). In addition, no significant difference in anti-CT IgA titers in serum were observed between the three groups of mice given the clone strain or each of the reference strains (FIG. 12).

### Example 6

### Data on Several Recombinant E. coli K12 Strains Co-Expressing Unnatural Combinations of ETEC CFs

The following plasmids were constructed, and then two of them, based on the desired combination, were electroporated into E. coli K12 bacteria TOP10.

pAF-CFA/I-Amp

pJT-CFA/I-Cm

pMT-CS2-Cm

pJT-CS4-Amp

pJT-CS6-Amp

The recombinant E. coli K12 strains:

TOP10-CFA/I-CS2

TOP10-CFA/I-CS6

TOP10-CS2-CS4

Demonstration of Co-Over-Expression of CFs on the Surface of Recombinant E. coli K12 Strains

To determine the co-expression of unnatural ETEC CFs on recombinant E. coli K12 strains, two different assays, i.e., dot blot and inhibition ELISA, were used. In the dot blot assay, 2 μl of two- or three-fold dilutions of whole bacteria in PBS in initial concentration of 10⁹ bacteria/ml, were applied on a nitrocellulose membrane. Following incubation of the membrane with corresponding anti-CF MAb (Table 2), the membrane was washed, incubated with anti-mouse IgG-horseradish peroxidase conjugate, and then developed (as described in Materials and Methods). Expression of CFs on the bacterial surface of recombinant and reference strains was also determined by use of inhibition ELISA as described in Inhibition ELISA for quantification of ETEC CFs/CS-factors.

By this assay, the capacity of the CF on the respective strain to inhibit binding of corresponding anti-CF MAbs to solid-phase-bound CF is determined, as described in Inhibition ELISA for quantification of ETEC CFs/CS-factors.

The dot blot results are listed in Table 9 and the inhibition ELISA results are listed in Table 10.

TABLE 1
List of the bacterial strains, plasmids and primers used

| Strains, plasmid | |
|---|---|
| and primers | Relevant characteristic |
| Bacterial strains: | |
| 325542-3 | ETEC (vaccine) reference strain, expressing CFA/I |
| 58R957 | ETEC reference strain, expressing CS2 |
| VM75688 | ETEC reference strain, expressing CS6 |
| SBL107 | Vaccine strain, expressing CS2 |
| SBL104 | Vaccine strain, expressing CS6 |
| E. coli TOP10 | E. coli K12, F⁻ lambda⁻ (Invitrogen) |
| TOP10-CFA/I | E. coli TOP10 expressing CFA/I |
| TOP10-thyA⁻-CFA/I | thya⁻ E. coli TOP10 expressing CFA/I |
| TOP10-CS2 | E. coli TOP10 expressing CS2 |
| TOP10-CS6 | E. coli TOP10 expressing CS6 |
| Plasmids: | |
| pAF-CFA/I-AMP | 8850 bp; amp^{r} |
| pAF-thyA-CFA/I-AMP | 10050 bp; amp^{r}, thyA⁺ |
| pAF-thyA-CFA/I | 8800 bp; thyA⁺ |
| Primers ^{a}: | |
| CFA/I-forward | 5′-CGGTCTCGAATTCTGATGGAAGCTCAGGAGG (SEQ ID NO: 1) |
| CFA/I-reverse | 5′-CGGTCTCAAGCTTTCTAGAGTGTTTGACTACTTGG (SEQ ID NO: 2) |
| CS2-forward | 5′-CGGTCTCGAATTCTTCTTGAAAGCCTCATGC (SEQ ID NO: 3) |
| CS2-reverse | 5′-CGGTCTCAAGCTTTTTACAGACTTGAACTACTAGG (SEQ ID NO: 4) |
| CS6-forward | 5′-CGGTCTCGAATTCTAATGGTGTTATATGAAGAAAAAATTG (SEQ ID NO: 5) |
| CS6-reverse | 5′-CGGTCTCAAGCTTAACATTGTTTATTTACAACAGATAATTGTTTG (SEQ ID NO: 6) |
| P1 | 5′-CGGTCTCTCATCTATTTCGGGAAGGCGTCTC (SEQ ID NO: 7) |
| P2 | 5′-CGGTCTCTGATGTTTTGGTTCCACTCAGCGTC (SEQ ID NO: 8) |

^{a} The primers were designed based on the CFA/I operon (GeneBank, accession number M55661), CS2 operon (GeneBank, accession number Z47800), CS6 operon (GeneBank, accession number U04846).

TABLE 2
Antigens and antibodies used in CF-inhibition ELISA

| | | | Monoclonal antibody (MAb)^{a} | | | |
|---|---|---|---|---|---|---|
| Colonization factor (CF) | | | | | Dilution used | |
| | Coating | Coating | | | in assay | |
| Deter | antigen | concen- | | | Inhi- | |
| mina- | for | tration | | Iso- | bition | Dot |
| tion of | ELISA | for ELISA | Designation | type | ELISA | Blot |
| CFA/I | CFA/I-18 | 1 μg/ml | CFA/I 1:6 | IgG1 | 1/200 | 1/50 |
| CS2 | CS2-18/7 | 1 μg/ml | CS2 10:3 | IgG1 | 1/200 | 1/50 |
| CS6 | rCS6 | 0.3 μg/ml | CS6 2a:14 | IgG1 | 1/100 | 1/50 |

^{a}MAbs are culture filtrates of cloned hybridoma cells (50-100 μg Ig/ml filtrate).

TABLE 3
Comparison of the relative amounts of CFs on the recombinant and
corresponding reference strains by the dot blot assay

| | Titer by dot blot assay | |
|---|---|---|
| Surface CF | E. coli recombinant strain | ETEC reference strain |
| CFA/I | 32 (10-52)* | 4.5 (2-7) |
| CS2 | 31 (29.5-32.5)* | 3.5 (2.8-4.2) |
| CS6 | 24 (15-33)* | 2.5 (1.3-3.7) |

^{a} The titers indicate the highest reciprocal dilution of bacteria in initial concentration 10⁹ bacteria/ml, giving a visible reaction with corresponding specific MAb; the values shown are arithmetic means of at least three experiments and range of mean ± SD; in the case of CS2, the results are the value of arithmetic mean of two experiments.
*show the significant differences (p < 0.05).

TABLE 4
Comparison of the reciprocal inhibitory titers of the recombinant and
corresponding reference strains by inhibition ELISA

| | Titer by inhibition ELISA ^{a} | |
|---|---|---|
| Surface CF | E. coli recombinant strain | ETEC reference strain |
| CFA/I | 595 (450-795)* | 135 (105-175) |
| CS2 | 67 (41-93)* | 4 (3.2-4.8) |
| CS6 | 30 (23-37)* | 5.7 (4.7-6.7) |

^{a} The titers are expressed as the reciprocal inhibitory dilution of each bacterial suspension, with initial concentration of 10⁹ bacteria/ml, which caused 50% inhibition of binding of the specific MAb to corresponding solid phase bound CF in ELISA as described in Inhibition ELISA for quantification of ETEC CFs/CS-factors. The values shown are arithmetic means of at least three experiments and ranges of mean ± SD.

TABLE 5
Comparison of the level of CFA/I on the ampicillin resistant and
ThyA dependent recombinant E. Coli strains

| | Reciprocal titer ^{a} | |
|---|---|---|
| Assay used for | Ampicillin resistant | ThyA dependent |
| detection of | TOP10-CFA/I recombinant | TOP10-CFA/I |
| CFA/I | strain | recombinant strain |
| Dot blot | 32 ^{b} | 32 ^{b} |
| Inhibition ELISA | 525 (490-560) ^{c} | 550 ^{c} |

^{a} The titers, in dot blot assay, indicate the highest reciprocal dilution of bacteria giving a visible and clear reaction with corresponding specific MAb. In inhibition ELISA, the titers are expressed as reciprocal inhibitory titer, which caused 50% inhibition of binding of the specific MAb to each CF, calculated as described in Inhibition ELISA for quantification of ETEC CFs/CS-factors. In both assays, the initial concentration of bacteria was 10⁹ bacteria/ml.
^{b} The results are the value of a single experiment.
^{c} The results are the value of duplicates in one experiment.

TABLE 6
Comparison of the CFA/I level on the recombinant TOP10-CFA/I
strain and on the CFA/I positive reference before and after
formalin inactivation

| | Level of CFA/I expression ^{a} | |
|---|---|---|
| Strain | Without treatment | With treatment |
| CFA/I over-expressing recombinant | 100% | 95.8% |
| E. coli strain | | |
| CFA/I reference strain (325542-3) | 100% | 97.8% |

^{a} The values indicate the level of CFA/I with and without inactivation, as measured by inhibition ELISA assay. The value for the CFA/I level without inactivation has been assigned 100 percent.

TABLE 7
Comparison of anti-CFA/I titers of, respectively, IgA and IgG + IgM
isotypes in mice orally immunized with formalin-inactivated bacteria
of the recombinant TOP10-CFA/I strain and the CFA/I positive
reference strain

| | Antibody titers against CFA/I, log 10 ^{a} | |
|---|---|---|
| | E. coli recombinant | ETEC reference strain |
| Isotype | strain | (325542-3) |
| IgA | 2.0 ± 0.33 * | 1.7 ± 0.11 |
| IgG + M | 3.9 ± 0.74 | 3.3 ± 0.57 |

^{a} The values indicate the reciprocal geometric mean of the log₁₀ titers ± SE based on two experiments with three mice in each for each tested strain.
* indicates significant difference (P < 0.05).

TABLE 8
List of strains, plasmids, and primers used

| Strains, plasmid | |
|---|---|
| and primers | Relevant characteristic |
| Strains: | |
| E. coli TOP10 | K12, F⁻ lambda⁻ |
| TOP10-pAF1-CFA/I | TOP10 expressing CFA/I, using pAF1 |
| TOP10-pAF1-CS2 | TOP10 expressing CS2, using pAF1 |
| TOP10-pMT-CS2 | TOP10 expressing CS2, using pMT |
| TOP10-CFA/I-CS2 | TOP10 expressing CFA/I and CS2, using pMT |
| Plasmid: | |
| pAF1-CFA/I | 8850 bp; amp^{r} |
| pAF1-CS2 | 8952 bp, amp^{r} |
| pMT-CS2 | 8746 bp; Cm^{r} |
| Primers: | |
| P1 SEQ ID NO: 9 | 5′-CGGTCTCGAATTCTTCTTGAAAGCCTCATGC |
| P2 SEQ ID NO: 10 | 5′-CGGTCTCAAGCTTTTTACAGACTTGAACTACTAGG |

TABLE 9
Dot blot results. Comparison of the relative amounts of CFs on the
recombinant and corresponding reference strains by the dot blot assay

| | Titer by dot blot assay ^{a} | | |
|---|---|---|---|
| Strains | anti CFA/I | anti CS2 | anti CS6 |
| SBL reference strain | 8 | 8 | <1 |
| TOP10-CFA/I | 64 | — | — |
| TOP10-CFA/I-CS2 | 32 | 16 | — |
| TOP10-CFA/I-CS6 | 32 | — | 16 |

| | Titer by dot blot assay ^{a} | |
|---|---|---|
| Strains | Anti CS2 | Anti CS4 |
| SBL reference wild-type strain | 8 | 8 |
| TOP10-CS2 | 32 | — |
| TOP10-CS2-CS4 | 32 | 32 |

^{a} The titers indicate the highest reciprocal dilution of bacteria in initial concentration 10⁹ bacteria/ml, giving a visible reaction with corresponding specific MAb.

TABLE 10
Inhibition ELISA results. Comparison of the reciprocal inhibitory
titers of the recombinant and corresponding reference strains by
inhibition ELISA.

| | Titer by inhibition ELISA ^{a} | | |
|---|---|---|---|
| Strains | anti CFA/I | anti CS2 | anti CS6 |
| Reference wild-type strain | 51 | 12 | <5 |
| TOP10-CFA/I | 395 | — | — |
| TOP10-CFA/I-CS2 | 295 | 35 | — |
| TOP10-CFA/I-CS6 | 195 | — | 37 |

| | Titer by inhibition ELISA ^{a} | |
|---|---|---|
| Strains | Anti CS2 | Anti CS2 |
| Reference wild-type strain | 12 | 13 |
| TOP10-CS2 | 58 | — |
| TOP10-CS2-CS4 | 10* | 150 |

^{a} The titers are expressed as the reciprocal inhibitory dilution of each bacterial suspension, with initial concentration of 10⁹ bacteria/ml, which caused 50% inhibition of binding of the specific MAb to corresponding solid phase bound CF in ELISA as described in Inhibition ELISA for quantification of ETEC CFs/CS-factors.
*Dot blot showed four-fold higher CS2 (on TOP10-CS2-CS4) compared to the reference wild-type strain in Table 9.

TABLE 11
Immunogenicity of formalin-inactivated recombinant E. coli K12
strains

| | Antibody titers; log 10 ^{a} | | | |
|---|---|---|---|---|
| | anti CFA/I | | anti CS2 | |
| | IgA | IgG + M | IgA | IgG + M |
| CFA/I reference strain | 2.2 ± 0.2 | 3.86 ± 0.72 | — | — |
| (325542-3) | | | | |
| CS2 reference strain | — | — | 2.05 ± 0.22 | 3.16 ± 0.36 |
| (58R957) | | | | |
| TOP10-CFA/I-CS2 | 2.26 ± 0.11 | 4.7 ± 0.15 | 1.89 ± 0.12 | 4.18 ± 0.18* |

^{a} The values indicate the reciprocal geometric mean of the log10 titers ± SEM based on two experiments with three mice in each for each tested strain.
*indicates significant difference (P < 0.05).

### REFERENCES

Ahren C., M. Jertborn, and A. M. Svennerholm (1998). Intestinal immune responses to an inactivated oral enterotoxigenic Escherichia coli vaccine and associated immunoglobulin A responses in blood. Infect. Immun. 66:3311-3316.
Gaastra W. and A. M. Svennerholm (1996). Colonization factors of human enterotoxigenic Escherichia coli (ETEC). Trends Microbiol. 4:444-452.
Jertborn M., C. Ahren, J. Holmgren, and A. M. Svennerholm (1993). Safety and immunogenicity of an oral inactivated enterotoxigenic Escherichia coli vaccine. Vaccine 16:255-60.
Levine M. M., J. A. Giron, and F. Noriega (1994). Fimbrial vaccines, in Fimbriae: adhesion, biogenics, genetics and vaccines, P. Klemm (ed.), pp. 255-270, CRC Press, Boca Raton, Fla.
Qadri F., A. M. Svennerholm, A. S. G. Faruque, and R. B. Sack (2005a). Enterotoxigenic Escherichia coli in developing countries: epidemiology, microbiology, clinical features, treatment, and prevention. Clin. Microbiol. Rev. 18:465-483.
Qadri F., T. Ahmed, F. Ahmed, Y. A. Begum, D. A. Sack, A. M. Svennerholm and the PTE Study Group (2005b). Reduced doses of oral killed enterotoxigenic Escherichia coli plus cholera toxin B subunit vaccine is safe and immunogenic in Bangladeshi infants 6-17 months of age: Dosing studies in different age groups. Vaccine (in press).
Rhagavan S., M. Hjulström, J. Holmgren and A. M. Svennerholm (2002). Protection against experimental Helicobacter pylori infection after immunization with inactivated H. pylori whole cell vaccines. Infect. Immun. 70:6383-6388.
Rudin A., M. M. McConnell, A. M. Svennerholm (1994). Monoclonal antibodies against enterotoxigenic Escherichia coli colonization factor antigen I (CFA/I) that cross-react immunologically with heterologous CFAs. Infect. Immun. 62:4339-4346.
Sack D. A., J. Shimko, O. Torres et al. (2002). Safety and efficacy of a killed oral vaccine for enterotoxigenic E. coli diarrhea in adult travelers to Guatemala and Mexico. 42nd Interscience Conference on Antimicrobial Agents and Chemotherapy, San Diego, Calif. Abstract.
Sadeghi H., S. Bregenholt, D. Wegmann, J. S. Petersen, J. Holmgren, and M. Lebens (2002). Genetic fusion of human insulin B-chain to the B-subunit of cholera toxin enhances in vitro antigen presentation and induction of bystander suppression in vivo. Immunology 106:237-45.
Sambrook J. and R. W. Russell (2001). Molecular cloning: A laboratory manual, 3rd ed., Cold Spring, N.Y.: Cold Spring Harbor Laboratory Press.
Sanchez J. and J. Holmgren (2005). Virulence factors, pathogenesis and vaccine protection in cholera and ETEC diarrhea. Curr. Opin. Immunol. 17:388-98.
Savarino S. J., F. M. Brown, E. Hall, S. Bassily, F. Youssef, T. Wierzba, L. Peruski, N. A. El-Masry, M. Safwat, M. Rao, M. Jertborn, A. M. Svennerholm, Y. J. Lee, and J. D. Clemens (1998). Safety and immunogenicity of an oral, killed enterotoxigenic Escherichia coli-cholera toxin B subunit vaccine in Egyptian adults. J. Infect. Dis. 177:796-799.
Savarino S. J., E. Hall, S. Bassily, F. M. Brown, F. Youssef, T. F. Wierzba, L. Peruski, N. A. El-Masry, M. Safwat, M. Rao, H. El Mohamady, R. Abu-Elyazeed, A. Naficy, A. M. Svennerholm, M. Jertborn, Y. J. Lee, and J. D. Clemens (1999). Oral, inactivated, whole cell enterotoxigenic Escherichia coli plus cholera toxin B subunit vaccine: results of the initial evaluation in children. J. Infect. Dis. 179:107-14.
Stacey K. A. and E. Simson (1965). Improved Method for the isolation of thymine-requiring mutants of Escherichia coli. J. Bacteriol. 90:554-555.
Svennerholm A.-M., Y. L. Vidal, J. Holmgren, M. M. McConnell, and B. Rowe (1988). Role of PCF8775 antigen and its coli surface subcomponents for colonization, disease, and protective immunogenicity of enterotoxigenic Escherichia coli in rabbits. Infect. Immun. 56:523-528.
Svennerholm A.-M., C. Wenneras, J. Holmgren, M. M. McConnell, and B. Rowe (1990). Roles of different coli surface antigens of colonization factor antigen II in colonization by, and protective immunogenicity of, enterotoxigenic Escherichia coli in rabbits. Infect. Immun. 58:341-346.
Svennerholm A.-M. and J. Holmgren (1995). Oral vaccines against cholera and enterotoxigenic Escherichia coli diarrhea. Adv. Exp. Med. Biol. 371B:1623-1628.
Svennerholm A.-M. and S. J. Savarino (2004). Oral inactivated whole cell B subunit combination vaccine against enterotoxigenic Escherichia coli. In New Generation Vaccines: 3rd ed. Eds: Levine M. M. et al., pp. 737-750, Marcel Decker, New York, N.Y.
Svennerholm A.-M. and D. Steele (2004). Progress in enteric vaccine development. In: Microbial-gut interactions in health and disease, Ed. M. Farthing. Best Pract. Res. Clin. Gastroenterol. 18:421-445.

1. An Escherichia coli strain genetically engineered to express from recombinant plasmids one or more colonization factors (CFs) associated with enterotoxigenic Escherichia coli bacteria (ETEC) in an increased amount compared to said CFs expressed by ETEC wild-type reference strains.

2. The E. coli strain according to claim 1, wherein said E. coli strain is a non-toxigenic E. coli strain.

3. The E. coli strain according to claim 1, wherein CFs are selected from the group consisting of CFA/I, CS1, CS2, CS3, CS4, CS5 and CS6.

4. The E. coli strain according to claim 1, wherein said strain expresses an unnatural combination of at least two different CFs.

5. The E. coli strain according to claim 4, wherein said strain expresses an unnatural combination of two different CFs selected from the group consisting of CFA/I+CS2, CFA/I+CS6 and CS2+CS4.

6. The E. coli strain according to claim 1, wherein said strain does not express an antibiotic resistance gene.

7. The E. coli strain according to claim 1, wherein said strain carries one or more complementable chromosomal deletions or mutations that are complemented by one or more plasmids.

8. A method of producing an Escherichia coli cell carrying recombinant plasmids capable of expressing one or more colonization factors (CFs) associated with enterotoxigenic Escherichia coli bacteria (ETEC) in an increased amount compared to said CFs expressed by ETEC wild-type reference strains, the method comprising the steps of:
assembling in a plasmid genes required for expression of ETEC CFs; a powerful non-ETEC promoter that controls the expression of the CFs; a selection marker for plasmid maintenance; an origin of replication for the plasmid; and optionally, a means regulating the level of expression of the CFs
so as to produce E. coli carrying recombinant plasmids capable of expressing one or more CFs associated with ETEC in an increased amount compared to CFs expressed by ETEC wild-type reference strains.

9. A composition for use against diarrhea, the composition comprising:
at least one Escherichia coli strain according to claim 1, together with pharmaceutically acceptable excipients, buffers, and/or diluents.

10. The composition of claim 9, wherein the pharmaceutically acceptable excipients, buffers, and/or diluents are selected for oral delivery of the composition.

11. The E. coli strain of claim 2, wherein CFs are selected from the group consisting of CFA/I, CS1, CS2, CS3, CS4, CS5, and CS6.

12. The E. coli strain of claim 2, wherein the E. coli strain expresses an combination of at least two different CFs selected from the group consisting of CFA/I+CS2, CFA/I+CS6, and CS2+CS4.

13. The E. coli strain of claim 2, wherein the E. coli strain does not express an antibiotic resistance gene.

14. The E. coli strain of claim 2, wherein the E. coli strain carries one or more complementable chromosomal deletions or mutations that are complemented by one or more plasmids.

15. The E. coli strain of claim 3, wherein the E. coli strain does not express an antibiotic resistance gene.

16. The E. coli strain of claim 3, wherein the E. coli strain carries one or more complementable chromosomal deletions or mutations that are complemented by one or more plasmids.

17. The E. coli strain of claim 4, wherein the E. coli strain does not express an antibiotic resistance gene.

18. The E. coli strain of claim 4, wherein the E. coli strain carries one or more complementable chromosomal deletions or mutations that are complemented by one or more plasmids.


SEQUENCE LISTING (10 sequences)

SEQ ID NO: 1 - DNA, 31 bp, Artificial sequence
  primer
cggtctcgaattctgatggaagctcaggagg

SEQ ID NO: 2 - DNA, 35 bp, Artificial sequence
  primer
cggtctcaagctttctagagtgtttgactacttgg

SEQ ID NO: 3 - DNA, 31 bp, Artificial sequence
  primer
cggtctcgaattcttcttgaaagcctcatgc

SEQ ID NO: 4 - DNA, 35 bp, Artificial sequence
  primer
cggtctcaagctttttacagacttgaactactagg

SEQ ID NO: 5 - DNA, 41 bp, Artificial sequence
  primer
cggtctcgaattctaatggtgttatatgaagaaaacaattg

SEQ ID NO: 6 - DNA, 45 bp, Artificial sequence
  primer
cggtctcaagcttaacattgtttatttacaacagataattgtttg

SEQ ID NO: 7 - DNA, 31 bp, Artificial sequence
  primer
cggtctctcatctatttcgggaaggcgtctc

SEQ ID NO: 8 - DNA, 32 bp, Artificial sequence
  primer
cggtctctgatgttttggttccactcagcgtc

SEQ ID NO: 9 - DNA, 31 bp, Artificial sequence; identical to SEQ ID NO: 3
  primer

SEQ ID NO: 10 - DNA, 35 bp, Artificial sequence; identical to SEQ ID NO: 4
  primer